(19) United States
(12) Patent Application Publication
(10) Pub. No.: US 2015/0217004 A1
(43) Pub. Date: Aug. 6, 2015

(54) CARBAMATE COMPOUNDS AND METHODS OF USE IN DISEASES OF THE NERVOUS SYSTEM

(71) Applicant: Treventis Corporation, Bryn Mawr, PA (US)

(72) Inventors: Ian MacDonald, Halifax (CA); Sultan Darvesh, Halifax (CA); Earl Martin, Halifax (CA); Ian Pottie, Halifax (CA)

(21) Appl. No.: 14/425,504

(22) PCT Filed: Sep. 4, 2013
(86) PCT No.: PCT/US13/58008

§ 371 Date: Mar. 3, 2015

Related U.S. Application Data
(60) Provisional application No. 61/697,114, filed on Sep. 5, 2012.

(51) Int. Cl. A61K 51/04; C07D 487/04; C07C 271/58

(52) U.S. Cl. CPC A61K 51/0446; C07C 271/58; A61K 51/0402; C07D 487/04; C07B 2200/05

(57) ABSTRACT

In general, among other things, compounds of Formula I are provided:

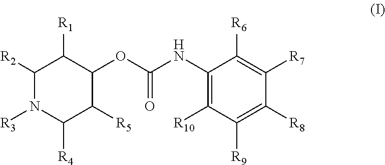

or a pharmaceutically acceptable salt thereof, in which R_{1-7, 9-10} are each independently selected from the group consisting of hydrogen, hydroxy, alkoxy, and alkyl; and R₈ is selected from the group consisting of fluoro, iodo, and tributyltin. Other compounds are also provided. Methods of treatment and diagnosis are also provided.

### BACKGROUND OF THE INVENTION

In Alzheimer's disease (AD) there are three major microscopic features in the brain that are recognized as the hallmarks of the disease, namely neuritic plaques (NP), neurofibrillary tangles (NFT) and amyloid angiopathy (AA). In addition, there is widespread cell loss, particularly of cholinergic neurons in the brain. Loss of cholinergic cells leads to reduction in the levels of the neurotransmitter acetylcholine, its synthesizing enzyme choline acetyltransferase, as well as its deactivating enzyme acetylcholinesterase (AChE, EC number 3.1.1.7). Reduction of cholinergic neurotransmission leads to some of the symptoms of AD.

Butyrylcholinesterase (BuChE) is found to have a brain distribution pattern that is distinct from that of acetylcholinesterase (AChE). Neurons containing BuChE are particularly located in the amygdala, hippocampal formation and the thalamus, structures involved in the normal functions of cognition and behavior that typically become compromised in Alzheimer's disease (AD). In the normal brain BuChE is mainly expressed in white matter, glia and distinct subcortical populations of neurons important for cognition and behavior. See e.g. S. Darvesh, D. L. Grantham, D. A. Hopkins, Distribution of butyrylcholinesterase in the humanamygdala and hippocampal formation, J Comp Neurol. 393 (1998) 374-390; S. Darvesh, D. A. Hopkins, Differential distribution of butyrylcholinesterase and acetylcholinesterase in the human thalamus, J Comp Neurol. 463 (2003a) 25-43; S. Darvesh, G. Geula, D. A. Hopkins, Neurobiology of butyrylcholinesterase, Nature Reviews Neuroscience. 4 (2003b) 131-138. AChE, in contrast, is found in neurons and neuropil throughout the brain. See e.g. M. Mesulam, C. Geula, Chemoarchitectonics of axonal and perikaryal acetylcholinesterase along information processing systems of the human cerebral cortex, Brain Res. Bull. 33 (1994) 137-153. In AD, both cholinesterases associate with Aβ plaques and NFTs. The accumulation of BuChE in AD pathology is especially notable in cortical grey matter, an area that normally has very little BuChE activity.

Although the level of AChE is reduced in AD, the level of the closely related enzyme butyrylcholinesterase (BuChE, EC number 3.1.1.8) is increased in AD brain. BuChE is found in the neuropathological lesions associated with AD, namely, NP, NFT and AA. Importantly, BuChE is found in NP in brains of patients with AD. BuChE is found in a higher number of plaques in brains of elderly individuals with AD relative to those without AD. It has been shown that some BuChE inhibitors not only improve cognition in an animal model but also reduce the production of β-amyloid, which is one of the principal constituents of neuritic plaques.

From a neuropathology perspective, deposition of amyloid and formation of NP is one of the central mechanisms in the evolution of AD. However, amyloid plaques are also found in brains of elderly individuals who do not have dementia (See, Guillozet et al., Butyrylcholinesterase in the life cycle of amyloid placques, Ann. Neurol. 42 (1997) 909-918.). It has been suggested that the amyloid plaques in individuals without dementia are “benign” and they become “malignant”, causing dementia, when they are transformed into plaques containing degenerated neurites. These plaques are called neuritic plaques (NP). The mechanism of transformation from “benign” to “malignant” plaques is as yet unknown. It has been suggested that BuChE may play a major role in this transformation based on the observation that BuChE is found predominately in plaques that contain dystrophic neurites and not in plaques without dystrophic neurites.

Taken together, these observations suggest that in the brains of patients with AD there is a significant alteration of the biochemical properties of BuChE that alters its normal regulatory role in the brain, thus contributing to the pathology of AD. A compound that can modulate BuChE would therefore be useful as a therapeutic or diagnostic for AD. There remains a need for such compounds.

Multiple sclerosis (MS) is a neuroinflammatory and neurodegenerative disease of the central nervous system. MS manifests as a progressive loss of physical and cognitive faculties thought to be a result of widespread demyelination within the brain. Current methods for diagnosis of MS rely upon presentation of clinical symptoms as well as MRI imaging of lesions in the brain. MRI is a sensitive approach for the visualization of MS lesions however, it remains a non-specific methodology and thus additional evidence is required to reach a diagnosis. There remains a need for MS-specific imaging agents in order to provide an early and definitive diagnosis of this disease. Early diagnosis is crucial as several disease modifying therapies have been proven effective for MS. BuChE has been observed to be associated with active MS lesions. See S. Darvesh et al., Butyrylcholinesterase activity in multiple sclerosis neuropathology, Chem. Biol. Interact. 187(2010) 425-431.

Primary brain tumours are the result of aberrant proliferation of brain cells. Tumours of this nature can cause an enormity of clinical symptoms dependent upon location and size within the brain. Several non-specific imaging approaches are used to visualize tumours, such as MRI. However, a method to specifically detect tumours at an early stage has not heretofore been reported.

The first cholinesterase inhibitor (ChEI) was introduced in 1997 and it has been known that certain chemical compounds such as carbamates had anticholinesterase activity even earlier. For example in 1995, it was known that “in cognitive responders, memory enhancement by physostigmine in Alzheimer's disease is correlated directly to the magnitude of plasma cholinesterase inhibition.” Sanjay Asthana MD, Clinical pharmacokinetics of physostigmine in patients with Alzheimer's disease Clinical Pharmacology & Therapeutics (1995) 58, 299-309.

Anticholinesterases such as the cholinergic drugs, donepezil, galantamine and the carbamate, rivastigmine, are now considered by many to be the first line pharmacotherapy for mild to moderate Alzheimer's disease enhance cognitive function and are known to act by enhancing cholinergic function in the brain. Birks J. Cholinesterase inhibitors for Alzheimer's disease, Cochrane Database of Systematic Reviews 2006, Issue 1, Art. No.: CD005593. DOI: 10.1002/14651858.CD005593. These drugs have slightly different pharmacological properties, but are thought to all work by inhibiting the breakdown of acetylcholine by blocking the enzyme acetylcholinesterase.

Alzheimer's patients often exhibit other symptoms including depression, anxiety and sleep disorders, all of which may benefit from treatment with acetylcholinesterase inhibitors, such as the carbamates rivastigmine and physostigmine.

However, the carbamate physostigmine has not been well tolerated by patients and therefore cannot be used clinically for treatment of Alzheimer's disease. One of the reasons for this is that physostigmine is an extremely powerful inhibitor of cholinesterases in that it deactivates both AChE and BuChE extremely fast, raising acetylcholine levels rapidly. For this reason, patients treated with physostigmine experience significant intolerable side effects.

Rivastigmine, on the other hand, deactivates cholinesterases slower relative to physostigmine, but still not slow enough to avoid side effects. The rivastigmine patch was developed to affect a slower release of the rivastigmine to overcome this problem and has been shown to have lessened the side effects because of slower rate of release of the drug and hence deactivation of cholinesterase.

Research to find carbamates having the desired anticholinesterase activity but with less of the undesirable characteristics of carbamates, such as high toxicity, narrow therapeutic window and short duration of action has continued. See e.g. Qian-Sheng Yu, Carbamate analogues of (-)-physostigmine: In vitro inhibition of acetyl-and butyrylcholinesterase, Feb Letter, Volume 234, Issue 1, 4 Jul. 1988, Pages 127-130; Maruyama W, Anti-apoptotic action of anti-Alzheimer drug, TV3326 [(N-propargyl)-(3R)-aminoindan-5-yl]-ethyl methyl carbamate, a novel cholinesterase-monoamine oxidase inhibitor. Neurosci Lett. 2003 May 8; 341(3):233-6.

According to U.S. Pat. No. 8,101,782, compounds which are hybrids of the carbamates rivastigmine and physostigmine may provide additive or synergistic therapeutic benefit, for example, for patients with Alzheimer's disease, Parkinson's disease, glaucoma, oncologic condition(s), or delayed gastric emptying, or patients suffering from attention deficit hyperactivity disorder (ADHD), phobia, stroke, multiple sclerosis, sleep disorders, psychiatric disorders, pain, anticholinergic drug overdose, or tobacco dependence i.e., use of the compounds in patients attempting smoking cessation. Although many carbamates (e.g. eptastigmine, quilostigmine, phenserine, tolserine) have been tested for their anticholinesterase activity, few have been effective and safe enough for use in treatment of patients (e.g.rivastigmine). \check{S}arka \check{S}t\check{e}pánková, Cholinesterases and Cholinesterase Inhibitors, Current Enzyme Inhibition, 2008, 4, 160-171.

Many of the above listed diseases are fatal using current medical practice. In none of these diseases is there any known, widely accepted therapy or treatment that can halt and/or reverse the aggregation of amyloid deposits and in many, diagnosis remains difficult. As such there remains an urgent need for treatments.

Early definitive AD diagnosis in the living brain is also urgently needed as it could greatly facilitate specific timely treatment of the disorder and the search for novel drugs to pre-empt progress of this disease. Radioligands have been developed to detect deposition of Aβ plaques in the brain; however, since many cognitively normal individuals also exhibit Aβ plaque deposition, this approach has inherent disadvantages for definitive AD diagnosis during life. The association of BuChE with Aβ plaques appears to be a characteristic of AD. This has prompted the search for radioligands that target BuChE in association with Aβ plaques that accumulate in cortical grey matter, a region normally with very little of this enzyme activity. A number of BuChE radioligands have been synthesized and preliminary testing indicates that some such radioligands enter the brain and accumulate in regions known to contain BuChE. Radioligands targeting unusual BuChE activity in the brain may represent a means for early diagnosis and treatment monitoring of AD.

### SUMMARY OF THE INVENTION

Carbamate compounds are disclosed which are butyrylcholinesterase inhibitors. Such compounds have particular utility in treatment of Alzheimer's disease and other amyloid diseases. Such compounds are also capable of being used as diagnostics for AD and other diseases in which alteration of quantities, location, or regulation of BuChE in brain may be diagnostic of a pathology.

The diphenyl carbamates of the present invention deactivate cholinesterases at slower rates that e.g. physostigmine and rivastigmine and therefore as therapeutic agents should have significantly fewer side effects relative to the existing carbamates. As diagnostic agents, given the slower rate of deactivation of cholinesterases, these carbamates should have better brain bioavailability to provide better molecular imaging of the brain.

In accordance with the above, the present invention provides compounds of Formula I:

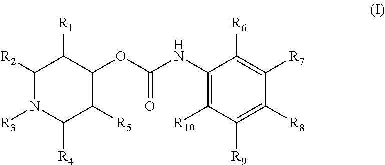

or a pharmaceutically acceptable salt thereof, in which R_{1-7,9-10} are each independently selected from the group consisting of hydrogen, hydroxy, alkoxy, and alkyl; and R₈ is fluoro or iodo.

The present invention also provides compounds of Formula II:

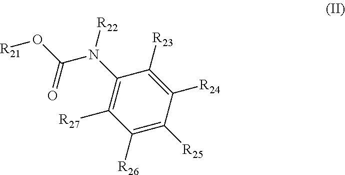

or a pharmaceutically acceptable salt thereof, in which R₂₁ is selected from the group consisting of phenyl, naphthyl, anthracenyl, phenanthrolinyl, adamantyl, indolyl, and N-alkylindolyl; R_{22,23,24,26,27} are each independently selected from the group consisting of hydrogen, hydroxy, alkoxy, and alkyl; and R₂₅ is fluoro or iodo.

The present invention also provides compounds of Formula III:

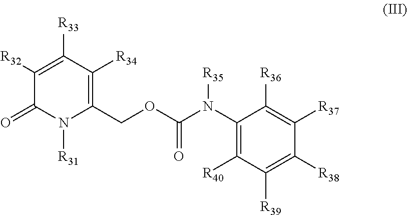

or a pharmaceutically acceptable salt thereof, in which R_{32-34, 36-37, 39-40} are each independently selected from the group consisting of hydrogen, hydroxy, alkoxy, and alkyl, and halogen; R₃₁ is selected from the group consisting of hydrogen, alkoxy, alkyl, and benzyl; R₃₅ is selected from the group consisting of hydrogen, alkoxy, and alkyl; and R₃₈ is fluoro or iodo.

The present invention also provides tributylstannyl intermediates (i.e., in which one or more halogen R groups can be replaced with SnBu₃).

The present invention also provides a method of treatment of an amyloid disease in a subject, including administering an effective amount of a compound of the present invention to the subject.

The present invention also provides a method of diagnosis of Alzheimer's disease in a subject, including administering an effective amount of a compound of the present invention to the subject.

The present invention also provides a method of diagnosis of multiple sclerosis in a subject, including administering an effective amount of a compound of the present invention to the subject.

The present invention also provides a method of diagnosis of brain tumour in a subject, including administering an effective amount of a compound of the present invention to the subject.

The present invention also provides a pharmaceutical composition having a compound of the present invention and a pharmaceutically acceptable excipient.

The present invention also provides a method for treating a condition which includes loss of memory, loss of cognition and a combination thereof, wherein the comprises administering to a subject in need thereof a therapeutically effective amount of a compound of Formula (I), Formula (II) or Formula (III) above. In certain embodiments, the condition is associated with Alzheimer's disease. The compound can be administered as a pharmaceutical composition comprising a pharmaceutically acceptable carrier. The total daily dose of the compound administered may be from about 0.0003 to about 30 mg/kg of body weight.

The present invention also provides a method of inhibiting butyrylcholinesterase activity in a patient which comprises administering to said patient a therapeutically effective amount of a compound of Formulas 1, II or III above.

The present invention also provides a method of treating a patient with Alzheimer's disease which comprises administering to said patient a therapeutically effective amount of a compound of Formula (I), Formula (II) or Formula (III) above.

The present invention also provides a method for treating an amyloid disease in a subject comprising administering to a subject in need thereof a therapeutically effective amount of a compound of Formulas I, II or III above. In certain embodiments, the amyloid disease may be Alzheimer's disease, Parkinson's disease or Huntington's disease.

In accordance with the above, the present invention is also directed to pharmaceutically acceptable salts, stereoisomers, polymorphs, metabolites, analogues, and pro-drugs of the compounds, and to any combination thereof.

With the foregoing and other advantages and features of the invention that will become hereafter apparent, the nature of the invention may be more clearly understood by reference to the following detailed description of the invention and the appended claims.

### BRIEF DESCRIPTION OF THE DRAWINGS

FIG. 1 shows the HPLC and radioactive count detection for the purification of phenyl 4-([¹²³I]iodo)phenylcarbamate, a compound of the present invention.

FIG. 2 shows interaction of phenyl 4-(iodo)phenylcarbamate with cholinesterases over time, with acetylcholinesterase (solid line, squares) and butyrylcholinesterase (dotted line, triangles) depicted.

FIG. 3. shows immunohistochemistry, histochemistry, or autoradiography as appropriate for Aβ, AChE, and BuChE in normal, normal-with-Aβ-plaques, and AD human brain tissue. Autoradiography of 4-([¹²³I]iodo)phenylcarbamate is depicted on the bottom row.

FIG. 4 shows immunohistochemistry, autoradiography, and a false color merged image for transgenic mouse brain tissue.

FIG. 5 shows histochemistry for luxol fast blue (LFB) and butyrylcholinesterase (BuChE) along with autoradiography with a cholinesterase (ChE) radioligand for post-mortem brain tissues from a MS patient.

FIG. 6 shows histochemical staining for butyrylcholinesterase (BuChE) and autoradiography with a cholinesterase (ChE) radioligand in biopsy tissue from a primary brain tumour.

FIG. 7 shows β-amyloid (Aβ) and butyrylcholinesterase (BuChE) staining in normal and Alzheimer's disease orbitofrontal cortex. Aβ plaques can be absent (A) or present (B) in brain tissue from cognitively normal brains. In AD, AB plaques are found extensively in the cortical grey matter. BuChE activity is largely absent in the grey matter of cognitively normal brain (D,E) but is found associated with plaques only in Alzheimer's disease (F). Therefore BuChE may distinguish between plaques in normal and AD brain.

FIG. 8 shows structures of cholinesterase substrate imaging agents.

FIG. 9 shows enzymatic trapping concept in which k₁ and k₂ represent the rates for uptake of the radioactive tracer into the brain and return back into the blood. kₐ represents the rate of formation of the enzyme-radioligand complex while k_{a′} designates the rate of dissociation of that complex. K3 corresponds to the very slow rate of egress of hydrophilic radioproduct from the brain.

FIG. 10 shows autoradiograms and butyrylcholinesterase (BuChE) histochemistry of wild type and Alzheimer's mouse. Ex vivo autoradiograms, after intravenous injection with a BuChE radioligand, demonstrate increased accumulation of radioactivity especially in cortical regions in the AD mouse (B) compared to wild type (A). This increased accumulation corresponds to the deposition of BuChE-positive plaques in the cortex of the AD mouse model (D) in contrast to the absence of BuChE in the cortex of the wild type mouse (C).

### DETAILED DESCRIPTION OF THE INVENTION

All patents, patent applications, and other publications referred to herein are hereby incorporated by reference in their entireties.

In one embodiment, compounds of Formula I are provided:

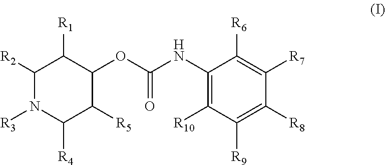

or a pharmaceutically acceptable salt thereof, in which R_{1-7, 9-10} are each independently selected from the group consisting of hydrogen, hydroxy, alkoxy, and alkyl; and R₈ is fluoro or iodo. In some embodiments, R₈ is ¹²³I or ¹⁸F.

In one embodiment, compounds of Formula II are provided:

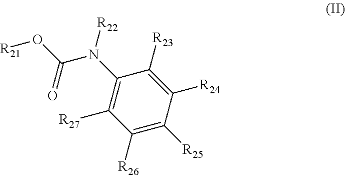

or a pharmaceutically acceptable salt thereof, in which R₂₁ is selected from the group consisting of phenyl, naphthyl, anthracenyl, phenanthrolinyl, adamantyl, indolyl, and N-alkylindolyl; R_{22,23,24,26,27} are each independently selected from the group consisting of hydrogen, hydroxy, alkoxy, and alkyl; and R₂₅ is fluoro or iodo. In some embodiments, R₂₅ is ¹²³I or ¹⁸F. A preferred diagnostic embodiment of a compound of Formula II is phenyl 4-([¹²³I]iodo)phenylcarbamate.

In one embodiment, compounds of Formula III are provided:

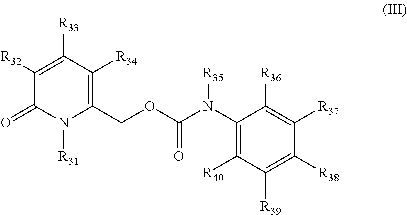

or a pharmaceutically acceptable salt thereof, in which R_{32-34,36-37,39-40} are each independently selected from the group consisting of hydrogen, hydroxy, alkoxy, and alkyl, and halogen;
R₃₁ is selected from the group consisting of hydrogen, alkoxy, alkyl, and benzyl; R₃₅ is selected from the group consisting of hydrogen, alkoxy, and alkyl; and R₃₈ is fluoro or iodo.

In one embodiment, tributylstannyl intermediates of compounds of Formulas I-III are provided, in which one or more halogen R groups is replaced with SnBu₃.

In one embodiment, a method of treatment of an amyloid disease in a subject is provided, comprising administering a therapeutically effective amount of a compound of the present invention to the subject. In some embodiments, the amyloid disease is Alzheimer's disease. In some embodiments, the amyloid disease is Parkinson's disease. In some embodiments, the amyloid disease is Huntington's disease.

In one embodiment, a method of diagnosis of Alzheimer's disease in a subject is provided, comprising administering a diagnostically effective amount of a compound of the present invention to the subject.

In one embodiment, a pharmaceutical composition is provided comprising a compound of the present invention and a pharmaceutically acceptable excipient.

It is believed that the compounds of the invention bind to BuChE and modulate it thereby. Data supportive of this conclusion can be found in the Examples below.

### DEFINITIONS

Unless otherwise defined, terms as used in the specification refer to the following definitions, as detailed below.

The terms “administration” or “administering” compound should be understood to mean providing a compound of the present invention to an individual in a form that can be introduced into that individual's body in an amount effective for prophylaxis, treatment, or diagnosis, as applicable. Such forms may include e.g., oral dosage forms, injectable dosage forms, transdermal dosage forms, inhalation dosage forms, and rectal dosage forms.

The term “alkoxy” as used herein means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkyl” as used herein means a straight or branched chain hydrocarbon containing from 1 to 20 carbon atoms, preferably from 1 to 10 carbon atoms, more preferably 1, 2, 3, 4, 5, or 6 carbons. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “carbonyl” as used herein means a —C(═O)-group.

The term “carboxy” as used herein means a —COOH group, which may be protected as an ester group: —COO-alkyl.

The term “fluoro” as used herein means —F.

The term “halo” or “halogen” as used herein means Cl, Br, I, or F.

The term “heteroaryl”, as used herein, refers to an aromatic ring containing one or more heteroatoms independently selected from nitrogen, oxygen, or sulfur, or a tautomer thereof. Such rings can be monocyclic or bicyclic as further described herein. Heteroaryl rings are connected to a parent molecular moiety through a carbon or nitrogen atom.

The terms “heteroaryl” or “5- or 6-membered heteroaryl ring”, as used herein, refer to 5- or 6-membered aromatic rings containing 1, 2, 3, or 4 heteroatoms independently selected from nitrogen, oxygen, or sulfur, or a tautomer thereof. Examples of such rings include, but are not limited to, a ring wherein one carbon is replaced with an O or atom; one, two, or three N atoms arranged in a suitable manner to provide an aromatic ring; or a ring wherein two carbon atoms in the ring are replaced with one O or S atom and one N atom. Such rings can include, but are not limited to, a six-membered aromatic ring wherein one to four of the ring carbon atoms are replaced by nitrogen atoms, five-membered rings containing a sulfur, oxygen, or nitrogen in the ring; five membered rings containing one to four nitrogen atoms; and five membered rings containing an oxygen or sulfur and one to three nitrogen atoms. Representative examples of 5- to 6-membered heteroaryl rings include, but are not limited to, furyl, imidazolyl, isoxazolyl, isothiazolyl, oxazolyl, pyrazinyl, pyrazolyl, pyridazinyl, pyridinyl, pyrimidinyl, pyrrolyl, tetrazolyl, [1,2,3]thiadiazolyl, [1,2,3]oxadiazolyl, thiazolyl, thienyl, [1,2,3]triazinyl, [1,2,4]triazinyl, [1,3,5]triazinyl, [1,2,3]triazolyl, and [1,2,4]triazolyl.

Heteroaryl groups of the invention can be substituted with hydrogen or alkyl. Monocyclic heteroaryl or 5- or 6-membered heteroaryl rings are substituted with 0, 1, 2, 3, 4, or 5 substituents. Heteroaryl groups of the present invention may be present as tautomers.

The term “hydroxy” as used herein means an —OH group.

Unless otherwise indicated, the term “prodrug” encompasses pharmaceutically acceptable esters, carbonates, thiocarbonates, N-acyl derivatives, N-acyloxyalkyl derivatives, quaternary derivatives of tertiary amines, N-Mannich bases, Schiff bases, aminoacid conjugates, phosphate esters, metal salts and sulfonate esters of compounds disclosed herein. Examples of prodrugs include compounds that comprise a biohydrolyzable moiety (e.g., a biohydrolyzable amide, biohydrolyzable carbamate, biohydrolyzable carbonate, biohydrolyzable ester, biohydrolyzable phosphate, or biohydrolyzable ureide analog). Prodrugs of compounds disclosed herein are readily envisioned and prepared by those of ordinary skill in the art. See, e.g., Design of Prodrugs, Bundgaard, A. Ed., Elseview, 1985; Bundgaard, hours., “Design and Application of Prodrugs,” A Textbook of Drug Design and Development, Krosgaard-Larsen and hours. Bundgaard, Ed., 1991, Chapter 5, p. 113-191; and Bundgaard, hours., Advanced Drug Delivery Review, 1992, 8, 1-38.

Unless otherwise indicated, the term “protecting group” or “protective group,” when used to refer to part of a molecule subjected to a chemical reaction, means a chemical moiety that is not reactive under the conditions of that chemical reaction, and which may be removed to provide a moiety that is reactive under those conditions. Protecting groups are well known in the art. See, e.g., Greene, T. W. and Wuts, P. G. M., Protective Groups in Organic Synthesis (3 rd ed., John Wiley & Sons: 1999); Larock, R. C., Comprehensive Organic Transformations (2 nd ed., John Wiley & Sons: 1999). Some examples include benzyl, diphenylmethyl, trityl, Cbz, Boc, Fmoc, methoxycarbonyl, ethoxycarbonyl, and pthalimido. Protecting groups include, for example, nitrogen protecting groups and hydroxy-protecting groups.

The term “sulfonyl” as used herein means a —S(O)₂-group.

The term “thioalkoxy” as used herein means an alkyl group, as defined herein, appended to the parent molecular moiety through a sulfur atom. Representative examples of thioalkoxy include, but are no limited to, methylthio, ethylthio, and propylthio.

The compounds of the invention can be used in the form of pharmaceutically acceptable salts derived from inorganic or organic acids. Pharmaceutically acceptable salt(s) are well-known in the art. For clarity, the term “pharmaceutically acceptable salts” as used herein generally refers to salts prepared from pharmaceutically acceptable non-toxic acids or bases including inorganic acids and bases and organic acids and bases. Suitable pharmaceutically acceptable base addition salts include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art. See, e.g., Remington's Pharmaceutical Sciences, 18 th ed. (Mack Publishing, Easton Pa.: 1990) and Remington: The Science and Practice of Pharmacy, 19th ed. (Mack Publishing, Easton Pa.: 1995). The preparation and use of acid addition salts, carboxylate salts, amino acid addition salts, and zwitterion salts of compounds of the present invention may also be considered pharmaceutically acceptable if they are, within the scope of sound medical judgment, suitable for use in contact with the tissues of humans and lower animals without undue toxicity, irritation, allergic response, and the like, are commensurate with a reasonable benefit/risk ratio, and are effective for their intended use. Such salts may also include various solvates and hydrates of the compound of the present invention.

Certain compounds of the present invention may be isotopically labelled, e.g., with various isotopes of carbon, fluorine, or iodine, as applicable when the compound in question contains at least one such atom. In preferred embodiments, methods of diagnosis of the present invention comprise administration of such an isotopically labelled compound.

Certain compounds of the present invention may exist as stereoisomers wherein, asymmetric or chiral centers are present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The invention contemplates various stereoisomers and mixtures thereof and these are specifically included within the scope of this invention. Stereoisomers include enantiomers and diastereomers, and mixtures of enantiomers or diastereomers. Individual stereoisomers of compounds of the invention may be prepared synthetically from commercially available starting materials which contain asymmetric or chiral centers or by preparation of racemic mixtures followed by resolution well known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

Certain compounds of the present invention may exist as cis or trans isomers, wherein substituents on a ring may attach in such a manner that they are on the same side of the ring (cis) relative to each other, or on opposite sides of the ring relative to each other (trans). Such methods are well known to those of ordinary skill in the art, and may include separation of isomers by recrystallization or chromatography. It should be understood that the compounds of the invention may possess tautomeric forms, as well as geometric isomers, and that these also constitute an aspect of the invention.

It should be noted that a chemical moiety that forms part of a larger compound may be described herein using a name commonly accorded it when it exists as a single molecule or a name commonly accorded its radical. For example, the terms “pyridine” and “pyridyl” are accorded the same meaning when used to describe a moiety attached to other chemical moieties. Thus, for example, the two phrases “XOH, wherein X is pyridyl” and “XOH, wherein X is pyridine” are accorded the same meaning, and encompass the compounds pyridin-2-ol, pyridin-3-ol and pyridin-4-ol.

The term “pharmaceutically acceptable excipient”, as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of one skilled in the art of formulations.

Unless otherwise indicated, the terms “prevent,” “preventing” and “prevention” contemplate an action that occurs before a patient begins to suffer from the specified disease or disorder, which inhibits or reduces the severity of the disease or disorder or of one or more of its symptoms. The terms encompass prophylaxis.

Unless otherwise indicated, a “prophylactically effective amount” of a compound is an amount sufficient to prevent a disease or condition, or one or more symptoms associated with the disease or condition, or prevent its recurrence. A prophylactically effective amount of a compound is an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

Unless otherwise indicated, a “diagnostically effective amount” of a compound is an amount sufficient to diagnose a disease or condition. In general, administration of a compound for diagnostic purposes does not continue for as long as a therapeutic use of a compound, and could be administered only once if such is sufficient to produce the diagnosis.

Unless otherwise indicated, a “therapeutically effective amount” of a compound is an amount sufficient to treat a disease or condition, or one or more symptoms associated with the disease or condition.

The term “subject” is intended to include living organisms in which disease may occur. Examples of subjects include humans, monkeys, cows, sheep, goats, dogs, cats, mice, rats, and transgenic species thereof.

The term “substantially pure” means that the isolated material is at least 90% pure, preferably 95% pure, even more preferably 99% pure as assayed by analytical techniques known in the art.

The pharmaceutical compositions can be formulated for oral administration in solid or liquid form, for parenteral intravenous, subcutaneous, intramuscular, intraperitoneal, intra-arterial, or intradermal injection, for or for vaginal, nasal, topical, or rectal administration. Pharmaceutical compositions of the present invention suitable for oral administration can be presented as discrete dosage forms, e.g., tablets, chewable tablets, caplets, capsules, liquids, and flavored syrups. Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Parenteral dosage forms can be administered to patients by various routes including subcutaneous, intravenous (including bolus injection), intramuscular, and intraarterial. Because their administration typically bypasses patients' natural defenses against contaminants, parenteral dosage forms are specifically sterile or capable of being sterilized prior to administration to a patient. Examples of parenteral dosage forms include solutions ready for injection, dry products ready to be dissolved or suspended in a pharmaceutically acceptable vehicle for injection, suspensions ready for injection, and emulsions. Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like, and suitable mixtures thereof), vegetable oils (such as olive oil) and injectable organic esters such as ethyl oleate, or suitable mixtures thereof. Suitable fluidity of the composition may be maintained, for example, by the use of a coating such as lecithin, by the maintenance of the required particle size in the case of dispersions, and by the use of surfactants. These compositions may also contain adjuvants such as preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Suspensions, in addition to the active compounds, may contain suspending agents, for example, ethoxylated isostearyl alcohols, polyoxyethylene sorbitol and sorbitan esters, microcrystalline cellulose, aluminum metahydroxide, bentonite, agar-agar, tragacanth, and mixtures thereof. If desired, and for more effective distribution, the compounds of the invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations also are prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, one or more compounds of the invention is mixed with at least one inert pharmaceutically acceptable carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract in a delayed manner. Examples of materials which can be useful for delaying release of the active agent can include polymeric substances and waxes.

Compositions for rectal or vaginal administration are preferably suppositories which can be prepared by mixing the compounds of this invention with suitable non-irritating carriers such as cocoa butter, polyethylene glycol or a suppository wax which are solid at ambient temperature but liquid at body temperature and therefore melt in the rectum or vaginal cavity and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents. Dosage forms for topical or transdermal administration of a compound of this invention include ointments, pastes, creams, lotions, gels, powders, solutions, sprays, inhalants or patches. A desired compound of the invention is admixed under sterile conditions with a pharmaceutically acceptable carrier and any needed preservatives or buffers as may be required. Ophthalmic formulation, ear drops, eye ointments, powders and solutions are also contemplated as being within the scope of this invention. The ointments, pastes, creams and gels may contain, in addition to an active compound of this invention, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, lactose, talc, silicic acid, aluminum hydroxide, calcium silicates and polyamide powder, or mixtures of these substances. Sprays can additionally contain customary propellants such as chlorofluorohydrocarbons.

Compounds of the invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the invention, stabilizers, preservatives, and the like. The preferred lipids are the natural and synthetic phospholipids and phosphatidylcholines (lecithins) used separately or together. Methods to form liposomes are known in the art. See, for example, Prescott, Ed., Methods in Cell Biology, Volume XIV, Academic Press, New York, N.Y., (1976), p 33 et seq.

Actual dosage levels of active ingredients in the pharmaceutical compositions of this invention can be varied so as to obtain an amount of the active compound(s) that is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

An effective amount of one of the compounds of the invention can be employed in pure form or, where such forms exist, in pharmaceutically acceptable salt form. Alternatively, the compound can be administered as a pharmaceutical composition containing the compound of interest in combination with one or more pharmaceutically acceptable carriers. It will be understood, however, that the total daily usage of the compounds and compositions of the invention will be decided by the attending physician within the scope of sound medical judgment. The specific effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; the risk/benefit ratio; drugs used in combination or coincidental with the specific compound employed; and like factors well known in the medical arts. For example, it is well within the skill of the art to start doses of the compound at levels lower than required to achieve the desired therapeutic effect and to gradually increase the dosage until the desired effect is achieved.

The total daily dose of the compounds of the present invention as administered to a human or lower animal may range from about 0.0003 to about 30 mg/kg of body weight. For purposes of oral administration, more preferable doses can be in the range of from about 0.0003 to about 1 mg/kg body weight. If desired, the effective daily dose can be divided into multiple doses for purposes of administration; consequently, single dose compositions may contain such amounts or submultiples thereof to make up the daily dose. For oral administration, the compositions of the invention are preferably provided in the form of tablets containing about 1.0, about 5.0, about 10.0, about 15.0, about 25.0, about 50.0, about 100, about 250, or about 500 milligrams of the active ingredient.

Diagnostic uses can be as probes which, in conjunction with non-invasive neuroimaging techniques such as magnetic resonance spectroscopy (MRS) or imaging (MRI), or gamma imaging such as positron emission tomography (PET) or single-photon emission computed tomography (SPECT), are used to identify neuritic plaques (NP). For in vivo imaging, detection instrument availability greatly affects selection of a given label. The type of instrument used will guide the selection of the radionuclide or stable isotope. For instance, the radionuclide chosen must have a type of decay detectable by a given type of instrument. Another consideration relates to the half-life of the radionuclide. The half-life should be long enough so that it is still detectable at the time of maximum uptake by the target, but short enough so that the host does not sustain deleterious radiation. The radiolabeled compounds of the present invention can be detected using gamma imaging wherein emitted gamma irradiation of the appropriate wavelength is detected. Methods of gamma imaging include, but are not limited to, SPECT and PET. After a sufficient time has elapsed for the compound to bind BuChE (in a range between 30 minutes and 48 hours, for example), the area of the subject under investigation is examined by routine imaging techniques such as MRS/MRI, SPECT, PET, and CT. The exact protocol will necessarily vary depending upon factors specific to the patient, as noted above, and depending upon the body site under examination, method of administration and type of label used.

Radiolabelled diagnostics, using e.g. both human post-mortem brain tissues as well as mouse animal model of Alzheimer's disease, can also be used as an in vitro methodology for rapidly screening for compounds that can detect butyrylcholinesterase activity associated with Alzheimer's disease using autoradiography as a specific in vitro screening system.

The following is a method of the present invention for the production of radioligands. Compounds with a leaving group such as a tributyl tin, triflates or tosylates are dissolved in an appropriate solvent. To exchange the leaving group for iodine, the compound is treated with the appropriate reagent to incorporate radio-iodide. The exchange for fluorine is performed using potassium fluoride. These reactions are carried out until the starting material has disappeared using TLC analysis. The solvent is then evaporated and the product dissolved in dichloromethane or methanol. The product is purified by SEP pak and/or HPLC. Radio-iodination involves substitution of the precursor with an appropriate leaving group. The chemical reagent grade radionuclides are commercially available (¹²³I NaI, ¹³¹I NaI) as sodium iodide in sodium hydroxide solution. Precursors for radio-iodination include molecules with leaving groups such as tributyl tin, triflate and tosylate derivatives. The radiolabeled molecules are meant to be used for enzymatic assessment and binding assays. ¹²³I Labeling may be performed using N-chlorosuccinimide, iodobead or iodogen as a free radical initiator. The precursor is dissolved in an appropriate solvent and incubated with ¹²³I sodium iodide. For radio-fluorination, ¹⁸F can be obtained as either the F or F₂ form, depending on the irradiation conditions within a cyclotron. One production method of ¹⁸F isotopes involves proton irradiation of [¹⁸O] H₂O to yield [¹⁸F] as an F anion. Using a protective synthesis box, reagents for the synthesis of [¹⁸F] FDG can be adapted by those skilled in the art to effect synthesis of radiolabelled compounds of the present invention, by use of [¹⁸F] anion on a triflate derivative of the molecule to be labeled. In some embodiments, ¹⁸F can be introduced from the corresponding nitro or quaternary amine (e.g., NMe₃⁺) by reaction with ¹⁸F and K2.2.2 in the presence of potassium carbonate and DMSO/acetonitrile as the solvent system.

### EXAMPLES

### Synthetic Methods

All non-aqueous reactions were carried out in flame-dried round bottom flasks under an inert atmosphere (nitrogen or argon), unless otherwise stated. Temperatures indicated refer to an external bath. All reactions were magnetically stirred.

All organic solvents were distilled and dried following known procedures. Reagents were purchased from various commercial suppliers and used without further purification. The starting materials are either commercially available or may be prepared from commercially available reagents using chemical reactions known in the art.

Analytical thin layer chromatography (TLC) was performed using Merck 60 F-254 silica gel pre-coated glass plates (0.25 mm). Visualization was effected by short wave UV illumination, and/or KMnO₄ or PMA dip followed by development on a hot plate. Flash column chromatography was performed according to the procedure developed by Still using Fischer silica gel (32-63 particle size).

Melting points were measured on a Fisher-Johns Melting Point Apparatus with uncorrected temperatures. Infrared spectra were recorded as Nujol mulls or as neat liquids between sodium chloride plates on a Nicolet Avatar 330 FT-IR spectrometer. Peaks are reported in wavenumbers (cm⁻¹). Nuclear magnetic resonance (NMR) spectra were recorded on a Bruker AVANCE 500, operating at 500.1 MHz for ¹H and 125.8 MHz for ¹³C, with CDCl₃ as solvent (unless otherwise stated). Chemical shifts are reported in ppm relative to SiMe4 as internal standard and coupling constants reported in Hz. Low-resolution mass spectra were obtained using an Agilent 6890N GC with an Agilent 6890N Electron Impact MS (Waldbronn, Germany) operating at 70 eV, with fragments reported as their m⁺/z ratio. High-resolution mass spectra were obtained with accurate mass positive-ion electrospray ionization measurements using a Bruker Daltonics microTOF with a flow rate of 2 μL/min, spray voltage of 4,500 V and tray temperature of 180° C. Optical rotations were measured on a Perkin-Elmer 241 polarimeter with CH₂Cl₂ as solvent, unless otherwise stated.

The following scheme can be used to access radiolabeled compounds of the present invention:

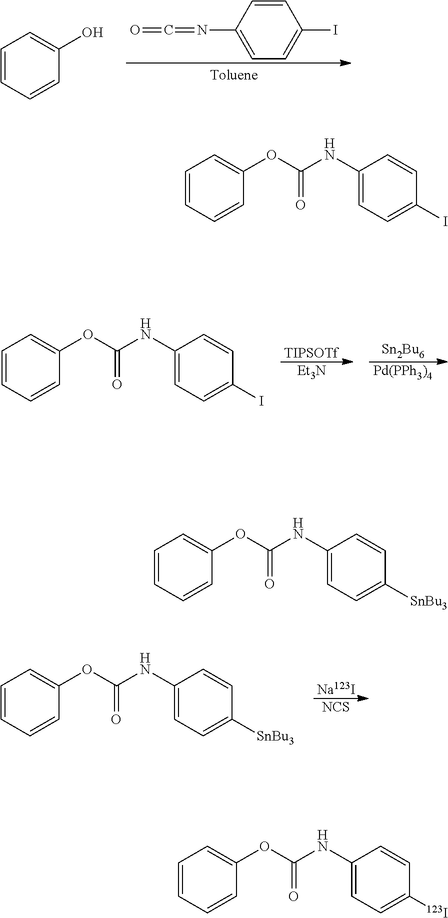

### Example 1

### Synthesis of Phenyl 4-(iodo)phenylcarbamate

Phenol (1.0206 g, 10.85 mmol) was dissolved in dry toluene (2 mL) under argon atmosphere and 4-iododiphenylisocyanate (2.6576 g, 10.85 mmol), dissolved in dry toluene (13 mL), was added to the solution. The reaction was refluxed for 5 hours, hot gravity filtered and the resulting white crystals collected (1.9833 g, 54%). Analytical data: MP: 159-161° C. (Lit: 160-162° C.). IR (Nujol) 3316, 1734, 1709, 1590, 1534, 1232 cm⁻¹. ¹H-NMR (CDCl₃): δ 6.95 (s, 1H), 7.19 (d, J=7.3 Hz, 2H), 7.24-7.28 (m, 3H), 7.41 (t, J=7.3 Hz, 2H), 7.65 (d, J=8.6 Hz, 2H) EI-MS m/z: 90 (21), 118 (11), 217 (2), 245 (100). HRMS (ESI): M⁺Na found 361.9648 calcd for C₁₃H₁₀INO₂Na=0.361.9654.

### Example 2

### Synthesis of Phenyl 4-(tributylstannyl)phenylcarbamate

Phenyl 4-(iodo)phenylcarbamate (0.2003 g, 0.59 mmol) was suspended in dry dichloromethane (10 mL) under argon atmosphere. Triethylamine (0.180 mL, 1.3 mmol) was added followed by triisopropylsilyltriflate (0.320 mL, 1.2 mmol). This solution was then added to tetrakis(triphenylphosphine) palladium (0.0262 g, 0.023 mmol) flowed by hexabutylditin (0.60 mL, 1.2 mmol). The resulting solution was refluxed for 16 hours and the solvent removed in vacuo. The resulting crude product was purified by silica gel column chromatography (1:9 ethyl acetate/hexanes) to yield a white solid (0.1474 g, 50%). Analytical data: MP: 53-55° C. IR (Nujol) 1719, 2852, 3331 cm⁻¹. ¹H-NMR (CDCl₃): δ 0.95 (t, J=7.3 Hz, 9H), 1.09-1.12 (m, 6H), 1.37 (sex, J=7.4 Hz, 6H), 1.55-1.60 (m, 6H), 6.90 (s, 1H), 7.19-7.25 (m, 3H), 7.39-7.44 (m, 6H). EI-MS m/z: 119 (9), 162 (9), 238 (98), 296 (60), 352 (100). HRMS (ESI): M⁺Na found 526.1738; calcd for C₂₅H₃₇NO₂Sn=526.1744.

### Example 3

### Synthesis of Phenyl 4-([¹²³I]iodo)phenylcarbamate

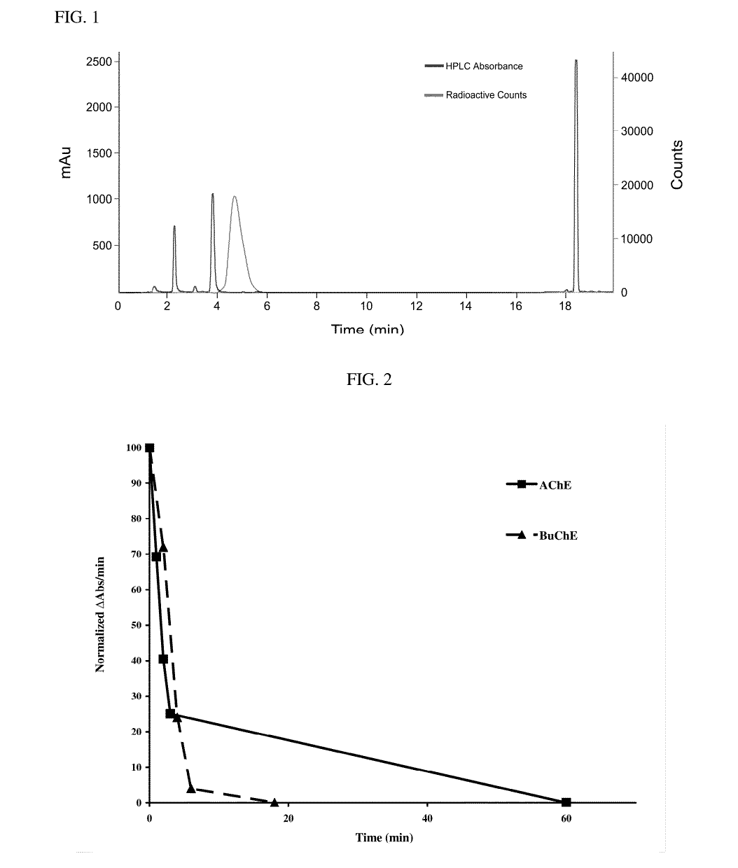

To a solution (9 μL) of Na¹²³I (64.42 MBq) in 0.1 M NaOH_{(aq)} (9.0×10 mol) was added NaI (3 μL, 5.5×10⁻⁹ mol) and 0.1 M HCl (18 μL, 1.8×10⁻³ mol) to neutralize the hydroxide. Phenyl 4-(tributylstannyl)phenylcarbamate (50 μL, 4.0×10⁻⁷ mol) was added to the solution followed by N-chlorosuccinimide (28 μL, 8.4×10⁻⁸ mol), both of which we dissolved in MeOH. The reaction proceeded for 15 min at room temperature; then 0.1 M NaHCO₃ (27 μL, 2.7×10⁻³ mol) was added to quench the reaction. Purification was accomplished by HPLC, using an Agilent system with a Zorbax Eclipse XDB-C18, 4.6×150 mm, 5 μm column (Agilent Technologies), and 1.0 mL/min of 80% MeOH_{(aq)} eluent. Fractions were collected every 20 sec for 15 min with a RediFrac fraction collector (Amershan Biosciences). Retention times were determined using the corresponding cold Phenyl 4-(iodo)phenylcarbamate as a non-radioactive standard. Collected fractions that contained purified product were combined and the solvent removed under a stream of N₂ gas with gentle heating to yield the desired radiolabelled compound as a residue (radiochemical yield 25%, as demonstrated in FIG. 1). The residue was dissolved either with 0.1 M maleate buffer pH 6.8, for incubation with tissue, or in 20% ethanol_{(aq)}, for animal administration.

### Example 4

### Synthesis of (3aR)-1,3a,8-trimethyl-1,2,3,3a,8,8a-hexahydropyrrolo[2,3-b]indol-5-yl 4-tributylstannylphenylcarbamate

(3aR)-1,3a,8-trimethyl-1,2,3,3a,8,8a-hexahydropyrrolo[2,3-b]indol-5-yl 4-iodophenylcarbamate (0.0795 g, 0.17 mmol) was dissolved in dry dichloromethane (5 mL) under argon atmosphere. Triethylamine (0.07 mL, 0.50 mmol) was added dropwise followed by triisopropylsilyl trifluoromethanesulfonate (0.13 mL, 0.48 mmol). This solution was added to Tetrakis(triphenylphosphine)palladium(0) (0.0126 g, 0.01 mmol) and hexabutylditin (0.34 mL, 0.68 mmol) was added dropwise. The reaction was refluxed for 16 hours in the dark and the solvent removed in vacuo. The resulting crude product was purified by silica gel column chromatography (1:20 MeOH/CH₂Cl₂) to produce a pink solid (0.0050 g, 47%). Analytical data: ¹H-NMR (CDCl₃): δ 0.94 (t, J=7.3 Hz, 9H), 0.97-0.99 (m, 1H), 1.08-1.11 (m, 6H), 1.13-1.16 (m, 2H), 1.37 (sex, J=7.3 Hz, 6H), 1.55-1.59 (m, 6H), 2.11-2.14 (m, 1H), 2.19-2.23 (m, 1H), 2.64 (s, 3H), 2.68-2.72 (m, 1H), 3.03 (m, 4H), 4.48 (s, 1H), 6.45 (d, J=8.2 Hz, 1H), 6.87 (d, J=2.4 Hz, 1H), 6.93 (dd, J=2.5, 8.2 Hz, 2H), 7.38-7.42 (m, 4H). ¹³C-NMR (CDCl₃): δ 9.8, 13.8, 13.9, 18.1, 26.9, 27.6, 29.2, 37.1, 37.5, 40.2, 53.2, 97.4, 107.5, 116.5, 118.5, 121.3, 137.0, 137.4, 137.5, 143.5, 149.2, 152.5.

### Example 5

### Synthesis of (3aR)-1,3a,8-trimethyl-1,2,3,3a,8,8a-hexahydropyrrolo[2,3-b]indol-5-yl 4-[¹²³I]iodophenylcarbamate

To a solution (9 μL) of Na¹²³I (64.42 MBq) in 0.1 M NaOH_{(aq)} (9.0×10 mol) was added NaI (3 μL, 5.5×10⁻⁹ mol), 0.1 M HCl (18 μL, 1.8×10⁻³ mol) to neutralize the hydroxide and 0.1 M NaHCO₃ (27 μL, 2.7×10⁻³ mol). (3aR)-1,3a,8-trimethyl-1,2,3,3a,8,8a-hexahydropyrrolo[2,3-b]indol-5-yl 4-tributylstannylphenylcarbamate (50 μL, 3.2×10⁻⁷ mol) was added to the solution followed by N-chlorosuccinimide (28 μL, 8.4×10⁻⁸ mol), both of which were dissolved in MeOH. The reaction proceeded for 15 min at room temperature. Purification was accomplished by HPLC, using an Agilent system with a Zorbax Eclipse XDB-C18, 4.6×150 mm, 5 μm column (Agilent Technologies), and 1.0 mL/min of 80% MeOH_{(aq)} eluent. Fractions were collected every 20 sec for 15 min with a RediFrac fraction collector (Amershan Biosciences). Retention times were determined using the corresponding cold (3aR)-1,3a,8-trimethyl-1,2,3,3a,8,8a-hexahydropyrrolo[2,3-b]indol-5-yl 4-iodophenylcarbamate as a non-radioactive standard. Collected fractions that contained purified product were combined and the solvent removed under a stream of N₂ gas with gentle heating to yield the desired radiolabelled compound as a residue (radiochemical yield 39%). The residue was dissolved either with 0.1 M maleate buffer pH 6.8, for incubation with tissue, or in 20% ethanol_{(aq)}, for animal administration.

### Example 6

### Synthesis of iodo diphenyl carbamates and fluoro diphenyl carbamates

The following compounds were synthesized using the procedures set forth in Examples 1-5 above.

### Synthesis of N-4-iodophenyl(9-phenanthryl)carbamate

9-phenanthrol (0.443 g, 2.28 mmol) was dissolved in anhydrous toluene (3 mL) under argon atmosphere and to this was added 4-iodophenyl isocyanate (0.559 g, 2.28 mmol) dissolved in anhydrous toluene (3 mL). The resulting mixture was refluxed overnight under argon. The reaction mixture was cooled to produce white crystals (0.429, 42%). Analytical data: MP: 197-198° C. IR (Nujol): 3292, 1708, 1519, 1462, 1377, 1235, 1074 cm⁻¹. ¹H NMR (DMSO): δ 7.46 (d, J=8.8 Hz 2H), 7.83 (s, 1H), 8.01 (d, J=9.2 Hz, 1H), 8.10 (d, J=7.4 Hz, 1H), 8.82 (d, J=8.0 Hz, 1H), 8.88 (d, J=7.4 Hz, 1H), 10.78 (s, 1H). ¹³C NMR (DMSO): δ 105.0 (1), 116.8 (0), 120.9 (1), 122.8 (1), 122.9 (1), 123.1 (1), 123.9 (1), 125.6 (0), 126.2 (0), 126.6 (1), 126.7 (0), 127.3 (1), 127.5 (1), 131.2 (0), 133.2 (0), 137.4 (1), 137.7 (1), 148.6 (0), 151.1 (0). HRMS (ESI): M⁺Na found 461.9949; calcd for C₂₁H₁₄O₂NINa=461.9961.

### Synthesis of N-4-iodophenyl(1-naphthyl)carbamate

1-napthol (0.370 g, 2.57 mmol) was dissolved in anhydrous toluene (5 mL) under argon atmosphere and to this was added 4-iodophenyl isocyanate (0.644 g, 2.63 mmol) dissolved in anhydrous toluene (5 mL). The resulting mixture was refluxed overnight under argon. The reaction mixture was cooled to produce white crystals (0.171, 17%). Analytical data: MP: 172-173° C. IR (Nujol): 3271, 1710, 1461, 1378 cm⁻¹. ¹H NMR (DMSO): δ 3.39 (s, 2H), 5.31 (s, 1H), 6.44 (d, J=8.8 Hz, 1H), 6.90 (dd, J=7.2 Hz, 1.2 Hz, 1H), 7.30 (m, 1H), 7.35 (m, 2H), 7.49 (m, 1H), 7.84 (d, J=8.3 Hz, 1H), 8.16 (d, J=8.3 Hz, 1H) 10.14 (s, 1H). ¹³C NMR (DMSO): δ 76.6 (0), 108.9 (1), 117.4 (1), 119.2 (1), 121.5 (1), 122.9 (1), 125.5 (1), 127.0 (1), 127.4 (1), 128.3 (1), 135.3 (0), 138.0 (1), 138.3 (1), 149.5 (0), 154.1 (0). HRMS (ESI): ArNa found 411.9785; calcd for C₁₇H₁₂O₂NINa=411.9805.

### Synthesis of N-4-iodophenyl(2-naphthol)carbamate

2-naphthol (0.375 g, 2.60 mmol) was dissolved in anhydrous toluene (3 mL) under argon atmosphere and to this was added 4-iodophenyl isocyanate (0.634 g, 2.59 mmol) dissolved in anhydrous toluene (4 mL). The resulting mixture was refluxed overnight under argon. The reaction mixture was cooled to produce white crystals. Analytical data: MP: 215-216° C. IR (Nujol): 3304, 1740, 1707, 1592, 1537, 1464, 1377, 1309, 1230, 816 cm⁻¹. ¹H NMR (DMSO): δ 7.44 (d, J=8.9 Hz, 2H) 7.46 (dd, J=8.7, 2.4 Hz, 1H) 7.57 (m, 2H) 7.72 (d, J=8.9 Hz, 2H) 7.81 (d, J=2.4 Hz, 1H) 8.00 (m, 3H) 10.53 (br. s., 1H). ¹³C NMR (DMSO): δ 86.50(0), 118.56(1), 120.66(1), 121.84(1), 125.71(1), 126.66(1), 127.44(1), 127.67(1), 129.26(1), 130.86(0), 133.39(0), 137.56(1), 138.58(0), 148.06(0), 151.74(0). HRMS (ESI): M⁺Na found 411.9805; calcd for C₁₇H₁₂O₂NINa=411.9805.

### Synthesis of N-4-iodophenyl(2-adamantyl)carbamate

2-adamantol (0.400 g, 2.62 mmol) was dissolved in anhydrous toluene (2 mL) under argon atmosphere and to this was added 4-iodophenyl isocyanate (0.636 g, 2.60 mmol) dissolved in anhydrous toluene (5 mL). The resulting mixture was refluxed overnight under argon. The reaction mixture was cooled to produce white crystals (0.391, 38%). Analytical data: MP: 198-199° C. IR (Nujol): 3302, 1699, 1589, 1530, 1462, 1377, 1304, 1239 cm⁻¹. ¹H NMR (CDCl₃): δ 1.59 (d, J=12 Hz, 2H), 1.72-1.82 (m, 4H), 1.82-1.90 (m, 4H), 2.01 (d, J=12.61 Hz, 2H), 2.08 (s, 2H), 4.92 (s, 1H), 6.66 (s, 1H), 7.19 (d, J=8.15 Hz, 2H), 7.60 (d, J=8.77 Hz, 2H). ¹³C NMR (CDCl₃): δ 26.9 (1), 27.2 (1), 31.8 (2), 32.0 (1), 36.3 (2), 37.3 (2), 78.3 (1), 86.0 (0), 120.4 (1), 137.9 (1), 138.0 (0), 152.9 (0). EI-MS m/z: 397 (26), 353 (32), 245 (82), 219 (14), 135 (100), 93 (21), 79 (24), 67 (16). HRMS (ESI): MH⁺found 420.0441; calcd for C₁₇H₂₀O₂NI=420.0431.

### Synthesis of N-4-iodophenyl(N-methyl-4-piperidinyl)carbamate

N-Methyl-4-piperidinol (0.321 g, 2.79 mmol) was dissolved in anhydrous toluene (3 mL) under argon atmosphere and to this was added 4-iodophenyl isocyanate (0.636 g, 2.89 mmol) dissolved in anhydrous toluene (3 mL). The resulting mixture was refluxed overnight under argon. The reaction mixture was cooled to produce white crystals (0.413, 41%). Analytical data: MP: 188-189° C. IR (Nujol): 3240, 1732, 1589, 1530, 1462, 1377, 1304, 1239 cm⁻¹. ¹H NMR (CDCl₃): δ 1.76 (m, 2H), 1.98 (m, 2H), 2.24 (s, 2H), 2.29 (s, 3H), 2.68 (s, 2H), 4.77 (s, 1H), 6.77 (s, 1H), 7.17 (d, J=8.5 Hz, 2H), 7.59 (m, 2H). ¹³C NMR (DMSO): δ 31.5 (2), 46.5 (3), 53.4 (2), 71.4 (0), 86.5 (0), 120.8 (1), 138.1 (1), 138.2 (1), 153.1 (0). EI-MS m/z: 360 (23), 245 (100), 218 (82), 219 (13), 98 (21), 97 (48), 90 (22). HRMS (ESI): MH⁺ found 361.0394; calcd for C₁₃H₁₈O₂N₂I=361.0407.

### Synthesis of phenyl (4-fluorophenyl)carbamate

Phenol (0.278 g, 2.96 mmol) was dissolved in anhydrous toluene (2 mL) under an anhydrous argon atmosphere. 4-Fluorophenyl isocyanate (0.33 mL, 2.94 mmol) was added and the resulting mixture was heated to reflux temperature for 6 hours. After this time, the reaction mixture was slowly cooled to room temperature. Clear colourless crystals precipitated upon cooling, the flask was placed in the freezer overnight to increase yield. Crystals were collected through suction filtration, washed with ice cold toluene (5 mL) and left to dry to afford 0.0696 g (10%) of phenyl (4-fluorophenyl)carbamate. Analytical data: MP: 160-161° C. IR (Nujol): 3364, 3312, 1734, 1715, 1615, 1555, 1217, 1101, 833, 766, 690 cm⁻¹. ¹H NMR (CDCl₃): δ 6.90 (br. s, 1H), 7.03 (t, J=8.9 Hz, 2H), 7.19 (d, J=8.6 Hz, 2H), 7.24 (t, J=7.5 Hz, 1H), 7.40 (m, 4H). ¹³C NMR (CDCl₃): δ 115.7, 115.9, 120.5, 121.6, 125.8, 129.4, 133.3, 150.5, 151.8. EI-MS m/z: 66, 82, 94, 109, 137, 231. HRMS (ESI): M⁺Na found 254.0581; calcd for C₁₃H₁₀FNNaO2=254.0593.

### Synthesis of 3-methoxyphenyl(4-fluorophenyl)carbamate

3-Methoxyphenol (0.32 mL, 2.92 mmol) was dissolved in anhydrous toluene (2 mL) under an anhydrous argon atmosphere. 4-Fluorophenyl isocyanate (0.33 mL, 2.94 mmol) was added and the resulting mixture was heated to reflux temperature for 6 hours. After this time, the reaction mixture was slowly cooled to room temperature. White crystals precipitated upon cooling, the flask was placed in the freezer overnight to increase yield. Crystals were collected through suction filtration, washed with ice cold toluene (5 mL) and left to dry to afford 0.369 g (49%) of 3-methoxyphenyl(4-fluorophenyl)carbamate. Analytical data: MP: 123-124° C. IR (Nujol): 3343, 1746, 1721, 1212, 1022, 852, 837, 672 cm⁻¹. ¹H NMR (CDCl₃): δ 3.80 (s, 3H), 6.74 (d, J=1.7 Hz, 1H), 6.78-6.80 (m, 2H), 6.94 (br. s, 1H), 7.02 (t, J=8.9 Hz, 2H), 7.28 (t, J=8.2 Hz, 1H), 7.39 (br. s, 2H).

### Synthesis of 4-methylphenyl(4-fluorophenyl)carbamate

p-Cresol (0.31 mL, 2.96 mmol) was dissolved in anhydrous toluene (2 mL) under an anhydrous argon atmosphere. 4-Fluorophenyl isocyanate (0.33 mL, 2.94 mmol) was added and the resulting mixture was heated to reflux temperature for 6 hours. After this time, the reaction mixture was slowly cooled to room temperature. White crystals precipitated upon cooling, the flask was placed in the freezer overnight to increase yield. Crystals were collected through suction filtration, washed with ice cold toluene (5 mL) and left to dry to afford 0.202 (28%) of 4-methylphenyl(4-fluorophenyl)carbamate. Analytical data: MP: 164-165° C. IR (Nujol): 3323, 1734, 1714, 1616, 1555, 1219, 1101, 830 cm⁻¹. ¹H NMR (CDCl₃): δ 2.35 (s, 3H), 6.87 (br. s, 1H), 7.01-7.07 (m, 4H), 7.18 (d, J=8.0 Hz, 2H), 7.39 (dd, J=7.8, 4.3 Hz, 2H).

### Biological Data

Esterase Activity Assay

The ability of the compounds to inhibit cholinesterases was evaluated by Ellman's spectrophotometric method (Ellman, G. L., Courtney, K. D., Andres, V. Jr. and Featherstone, R. M. A new and rapid colorimetric determination of acetylcholinesterase activity. Biochemical Pharmacology, 7, 88-95 (1961)) using human recombinant AChE and. acetylthiocholine as the substrate, and human serum BuChE with butyrylthiocholine as the substrate.

To test for enzyme deactivation over time, loss of enzyme activity was monitored. Briefly, 1.35 mL of buffered DTNB solution (pH 8.0), 0.05 mL of enzyme (0.03 units of human recombinant AChE or 0.05 units of purified human serum BuChE in 0.1% aqueous gelatin containing 0.01% sodium azide) and 0.05 mL of the carbamate dissolved in 50% aqueous acetonitrile at 0.5-5 mM, depending on solubility, were combined in a stoppered cuvette of 1 cm path length. After mixing and bringing the absorbance to zero, 0.05 mL of 4.8 mM aqueous acetyl- or butyrylthiocholine substrate solution was added to the cuvette after incubation of the enzyme with carbamate and buffered DTNB for periods of 30 min or longer. A zero-time sample was also obtained by adding enzyme last to initiate reaction and the second-order rate constants for enzyme deactivation (kₐ values) were determined. The kₐ value was calculated by plotting ln (e₀/eₜ)/[I] against time, where e₀ is the enzymatic activity at time zero (without preincubation of enzyme and inhibitor), eₜ is the enzymatic activity at time t min of preincubation, and [I] is the molar concentration of inhibitor. The slope of this plot gave the second-order rate constant. Experiments were generally done at least in triplicate and the values averaged.

The results of the effect on esterase activity by phenyl 4-(iodo)phenylcarbamate are shown in FIG. 2. This compound clearly has inhibitory effects on both AChE and BuChE, but on different timelines. Enzymatic deactivation proceeded rapidly indicating interaction of the ligand with both cholinesterase enzymes. This interaction, representing ligand bound to enzyme, persisted for hours and thus, is expected to provide an acceptable timeframe for imaging of the living brain.

Ex Vivo Autoradiography with Mouse Tissue

Mice were placed subjected to a continous flow of isoflurane to achieve anaesthetic conditions. A catheter was inserted into the mouse tail vein and 150 μL of phenyl 4-([123I]iodo)phenylcarbamate (13 MBq) was injected. After 30 min, the animal was sacrificed with sodium pentobarbitol (0.3 mL) and perfused transcardially with saline (50 mL) followed by 4% paraformaldehyde. The brain was immediately removed and placed in 4% paraformaldehyde for 30 min. The brain was frozen with dry ice and cut into 50 coronal sections. Every fourth section was immediately mounted on a glass slide and dried. The mounted tissue was exposed to a phosphorimaging screen (GE Healthcare) for 12 hours. The screen was scanned with a typhoon 9400 imager (GE Healthcare) to produce the autoradiogram. Image contrast was adjusted with Adobe Photoshop CS5.

In Vitro Autoradiography with Human Tissues

Human tissue from Alzheimer's disease or cognitively normal individuals was obtained from the Maritime Brain Tissue Bank (Halifax, Canada). These tissues were adjacent 50 coronal sections through areas such as the orbitofrontal cortex. The tissues were mounted on glass slides, in maleate buffer pH 6.8, and gently heated on a slide warmer until firmly adhered. The mounted tissue was rehydrated twice for 5 min, in maleate buffer pH 6.8, before placed in a coplin jar. Nine sections occupied each jar. 25 mL of maleate buffer pH 6.8 containing Phenyl 4-([¹²³I]iodo)phenylcarbamate (3.7 MBq) was added to the coplin jar and the tissue incubated for 18 hours at 37° C. with gentle agitation. At the completion of incubation, the tissue was rinsed twice for 1 min in distilled water and the slides dried on a slide wanner. The tissue was exposed to a high resolution phosphorimaging screen (GE Healthcare) for 22.5 hours. The screen was scanned with a typhoon 9400 imager (GE Healthcare) to produce the autoradiogram. Image contrast was adjusted with Adobe Photoshop CS5.

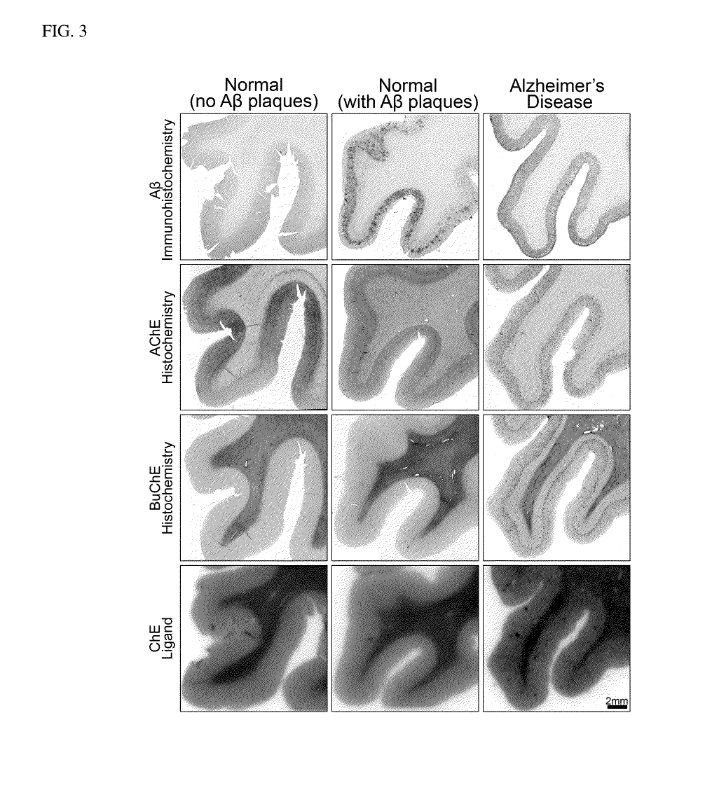

FIG. 3 shows the results of this autoradiography at bottom, compared to (immuno)histochemistry staining for Aβ, AChE, and BuChE. It can been seen that in the case of Aβ and AChE, the distinction between normal brain containing Aβ plaques and brains of known AD patients is unclear; thus from a diagnostic standpoint, those features alone are unlikely to be useful. BuChE histochemistry shows a clear distinction in the periphery of the tissue, in that there are many more stains in AD than in non-AD or non-AD-with-Aβ-plaque brain. However, such histochemistry must be performed in situ on the tissue once it is removed, which makes it undesirable as a diagnostic for AD. The bottom row showing the autoradiography for Phenyl 4-([¹²³I]iodo)phenylcarbamate demonstrates that normal-with-Aβ-plaques and AD brain tissue can be distinguished using compounds of the present invention; moreover, since the compound can be administered to a living subject, the compound has utility as a diagnostic for AD.

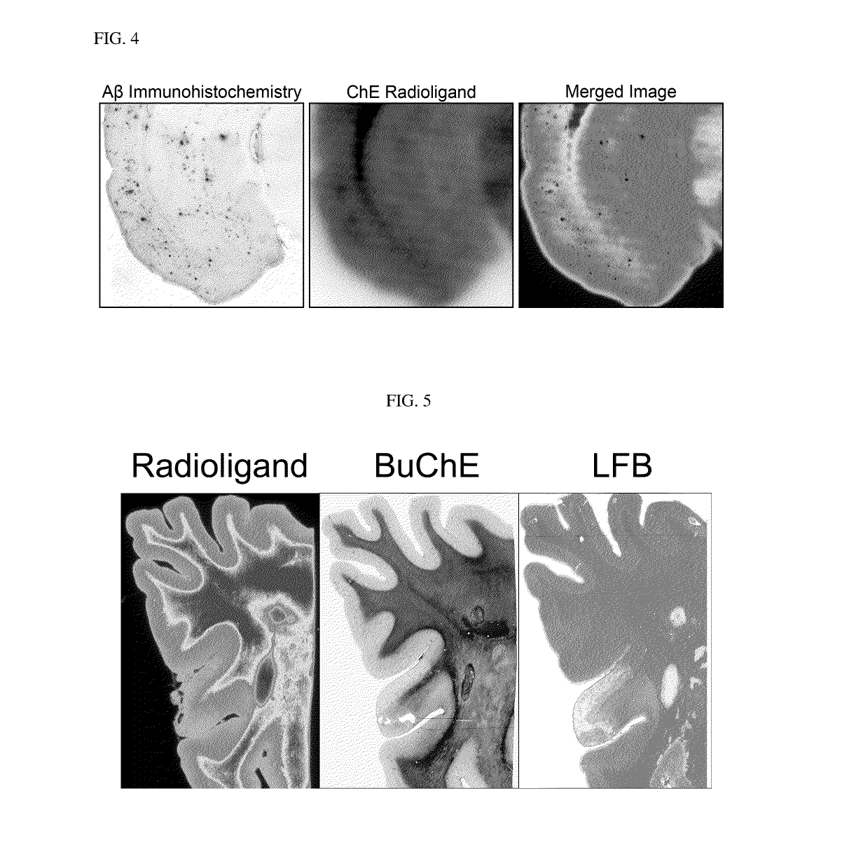

In vitro autoradiography using an AD transgenic mouse, FIG. 4, shows results which are consistent with those seen in FIG. 3. Some Aβ plaques, visualized by Aβ immunohistochemistry and similar to those in the human condition, were labelled by the radioligand compounds of the present invention.

For multiple sclerosis and brain tumour, human tissue from individuals with multiple sclerosis or primary brain tumours was obtained from the Maritime Brain Tissue Bank (Halifax, Canada). These tissues were adjacent 50 μm coronal sections. The tissues were washed twice, in maleate buffer pH 6.8, and to each section was added Phenyl 4-([¹²³I]iodo)phenylcarbamate (0.5 MBq). The tissue was incubated for 18 hours at 37° C. with gentle agitation. At the completion of incubation, the tissue was rinsed twice for a total of 15 min in distilled water and mounted on glass slides. Once dried, the tissue was exposed to a high resolution phosphorimaging screen (GE Healthcare) for 21 hours. The screen was scanned with a typhoon 9400 imager (GE Healthcare) to produce the autoradiogram. Image contrast was adjusted with Adobe Photoshop CSS.

FIG. 5 shows histochemistry for luxol fast blue (LFB) and butyrylcholinesterase (BuChE) along with autoradiography with a cholinesterase radioligand for post-mortem brain tissues from a MS patient. Autoradiography using this cholinesterase radioligand recapitulates the distribution of myelin, as can be seen with LFB, and BuChE in MS brain tissue. Thus, this radioligand demonstrates areas of aberrant BuChE activity indicative of MS lesions in this tissue. The radioligands presented in this application have the ability to detect cholinesterase activities in MS brain tissue and therefore, may provide diagnosis of this disease.

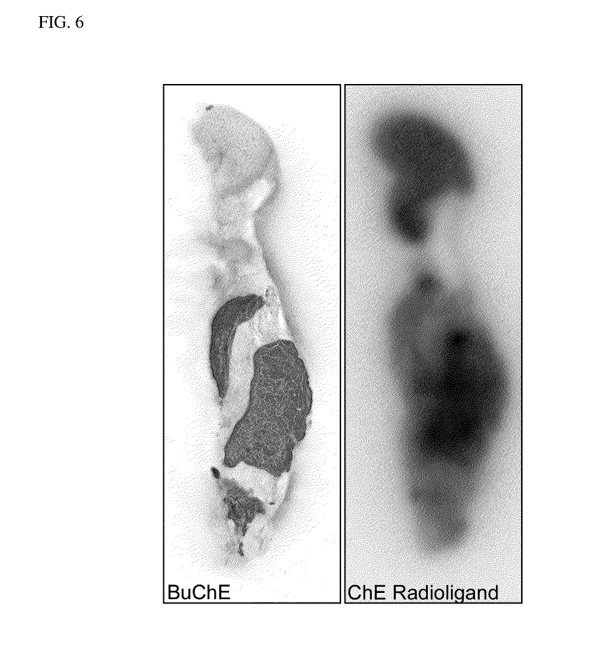

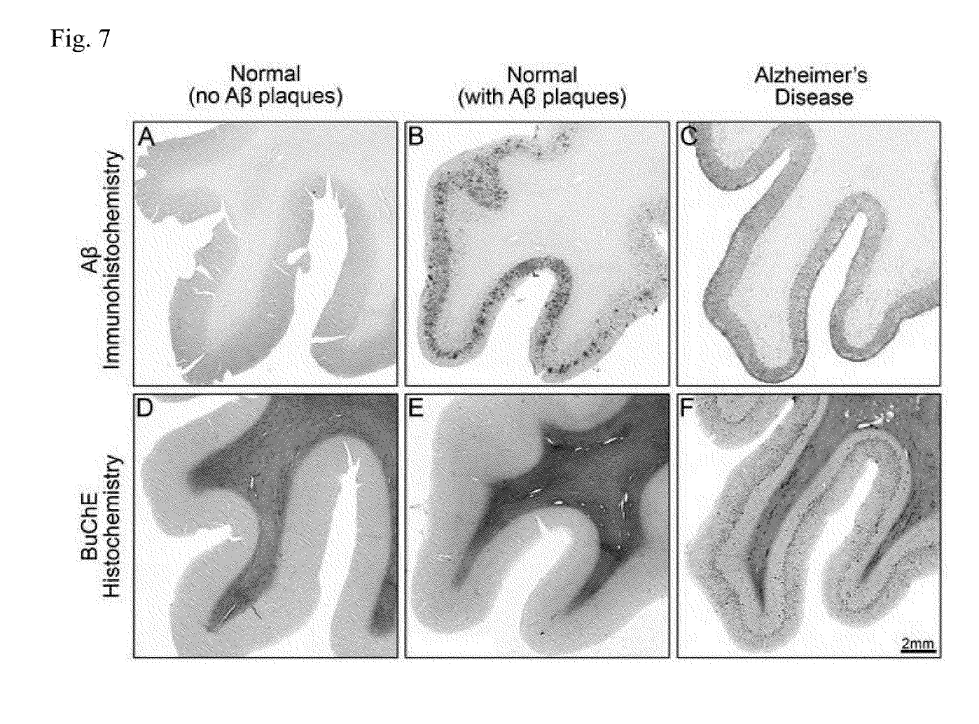

FIG. 6 shows histochemical staining for butyrylcholinesterase (BuChE) and autoradiography with a cholinesterase (ChE) radioligand in biopsy tissue from a primary brain tumour. Areas of high BuChE activity possessed significant accumulation of radioligand. Thus, areas of BuChE activity associated with primary brain tumours can be visualized with the radioligands presented in this application. These radioligands may detect cholinesterase activity associated with primary brain tumours in vivo and thus provide an early and definitive diagnosis of this condition.

Enzymatic Trapping

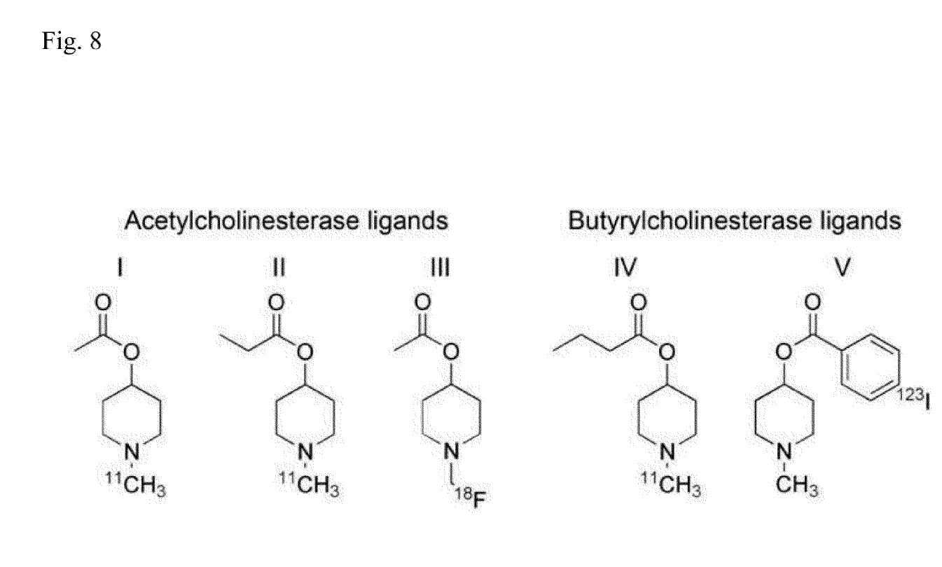

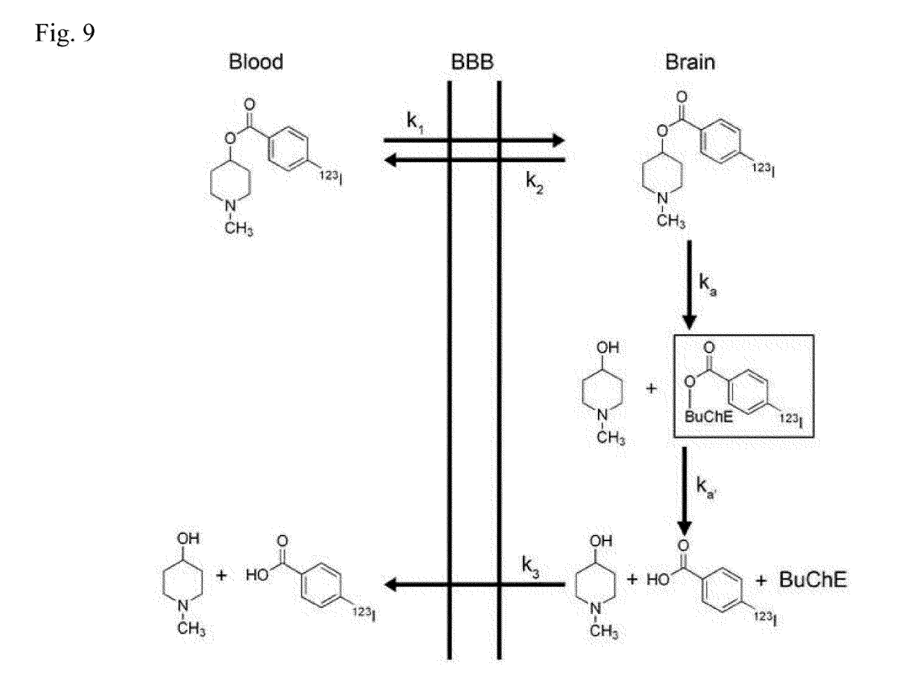

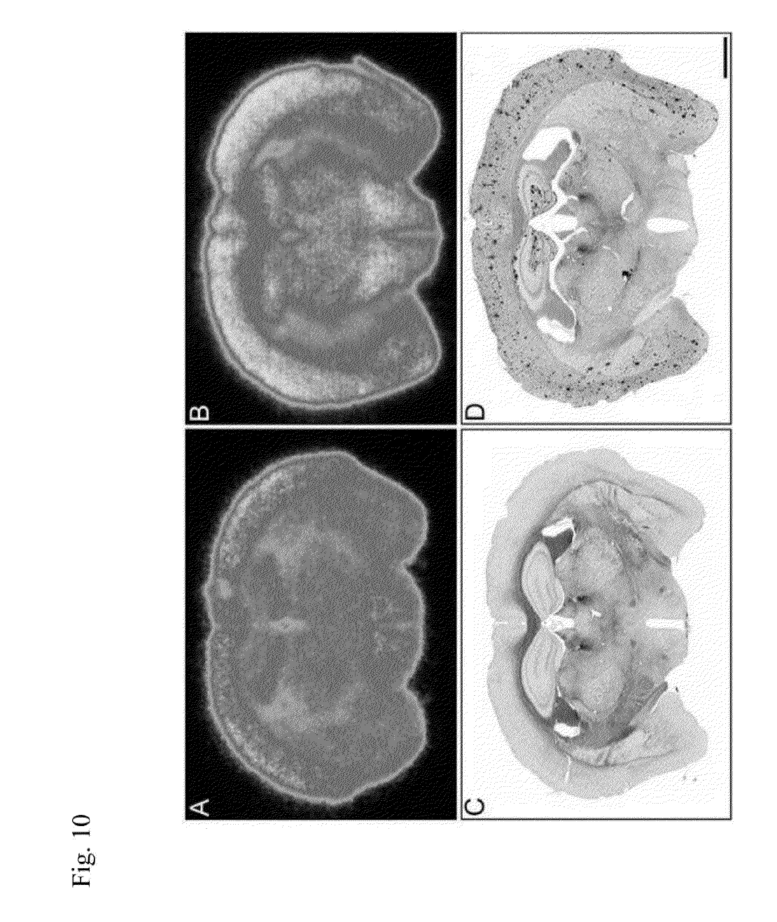

A radioactive atom is incorporated in the portion of a BuChE ligand, such as compound V (FIG. 8), that remains as part of the longer-lived acyl enzyme intermediate. In this concept, a more stable radiolabeled complex should facilitate accurate location of BuChE activity in the brain. As with metabolic trapping, a series of rate constants govern the retention of radiolabel in the brain (FIG. 9) where k1 and k2 represent the rates for uptake of the radioactive tracer into the brain and its return into the blood. The rate of formation of the enzyme-radioligand complex is represented by the constant ka, while ka′ corresponds to the rate of dissociation of that complex. The very slow rate of egress of hydrophilic radioproduct from the brain into the blood is designated k3. To test this enzyme trapping concept, a number of compounds were synthesized with 123I as part of the acyl group of the ester. Injection of such a compound (V in FIG. 8) intravenously into a rat model has shown that this tracer is able to accumulate, for the most part, in areas of the brain known to contain high levels of BuChE. Furthermore, injection of such tracers into an animal model of AD, in which there is substantially increased BuChE activity in the cerebral cortex associated with Aβ plaques showed that, relative to wild type, there was an increased accumulation of radioactivity in this area (FIG. 10). Histochemical analysis of the same brain indicated that, compared to wild type, there is substantially increased BuChE activity in the cerebral cortex (FIG. 10).

Carbamates, in particular, are important in enzymatic trapping because they can carbamylate the cholinesterase catalytic site. This enzyme trapping process retains the portion of a carbamate that contains the radiotracer atom and therefore these agents are better diagnostic agents than those previously described in the literature. This is particularly important for AD diagnosis as butyrylcholinesterase activity can distinguish plaques in AD from those found in cognitively normal older adults with plaques.

1. (canceled)

2. A compound of Formula II: 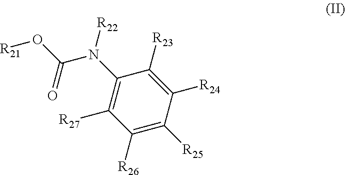
or a pharmaceutically acceptable salt thereof, in which R₂₁ is selected from the group consisting of phenyl, naphthyl, anthracenyl, phenanthrolinyl, adamantyl, indolyl, and N-alkylindolyl; R_{22,23,24,26,27} are each independently selected from the group consisting of hydrogen, hydroxy, alkoxy, and alkyl; and R₂₅ is selected from the group consisting of fluoro, iodo, and tributyltin.

3-4. (canceled)

5. The compound of claim 2 in which R₂₅ is ¹²³I or ¹⁸F.

6-8. (canceled)

9. A method of treatment of an amyloid disease in a subject comprising administering a therapeutically effective amount of a compound of claim 2 to the subject.

10. A method of diagnosis of Alzheimer's disease in a subject comprising administering a diagnostically effective amount of a compound of claim 2 to the subject.

11. The method of claim 9 in which the amyloid disease is Alzheimer's disease.

12. The method of claim 9 in which the amyloid disease is Parkinson's disease.

13. A method of diagnosis of multiple sclerosis in a subject comprising administering a diagnostically effective amount of a compound of claim 2 to the subject.

14. A method of diagnosis of brain tumour in a subject comprising administering a diagnostically effective amount of a compound of any of claims 1-8 to the subject.

15. A pharmaceutical composition comprising a compound of claim 2 and a pharmaceutically acceptable excipient.

16. A method for treating a condition which is a member selected from loss of memory, loss of cognition and a combination thereof, said method comprising administering to a subject in need thereof a therapeutically effective amount of a compound selected from the group consisting of Formula (II):
Formula II: 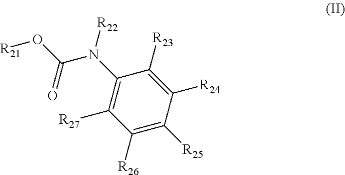
or a pharmaceutically acceptable salt thereof, in which R₂₁ is selected from the group consisting of phenyl, naphthyl, anthracenyl, phenanthrolinyl, adamantyl, indolyl, and N-alkylindolyl; R_{22,23,24,26,27} are each independently selected from the group consisting of hydrogen, hydroxy, alkoxy, and alkyl; and R₂₅ is selected from the group consisting of fluoro, iodo, and tributyltin.

17. The method according to claim 16, wherein said condition is associated with Alzheimer's disease.

18. The method according to claim 16, wherein said compound is administered as a pharmaceutical composition comprising a pharmaceutically acceptable carrier.

19. The method according to claim 18, wherein a total daily dose of from about 0.0003 to about 30 mg/kg of body weight is administered.

20. A method of inhibiting butyrylcholinesterase activity in a patient which comprises administering a therapeutically effective amount of a compound of claim 2 to the patient. A method of treatment of an amyloid disease in a subject comprising administering a therapeutically effective amount of a compound of claim 2 to the subject.

21. (canceled)

22. A method for treating an amyloid disease in a subject comprising administering to a subject in need thereof a therapeutically effective amount of a compound selected from the group consisting of Formula (II):
Formula II: 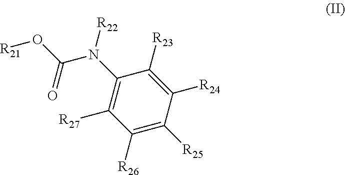
or a pharmaceutically acceptable salt thereof, in which R₂₁ is selected from the group consisting of phenyl, naphthyl, anthracenyl, phenanthrolinyl, adamantyl, indolyl, and N-alkylindolyl; R_{22,23,24,26,27} are each independently selected from the group consisting of hydrogen, hydroxy, alkoxy, and alkyl; and R₂₅ is selected from the group consisting of fluoro, iodo, and tributyltin.

23. The method according to claim 22, wherein said amyloid disease is Alzheimer's disease.

24. The method according to claim 22, wherein said amyloid disease is Parkinson's disease.

25. The method according to claim 22, wherein said amyloid disease is Huntington's disease.
